(19) United States
(12) Patent Application Publication
(10) Pub. No.: US 2024/0225898 A1
(43) Pub. Date: Jul. 11, 2024

(54) COUPLING INTERFACE BETWEEN A LASER SOURCE AND A TISSUE TO BE TREATED

(71) Applicant: KERANOVA, SAINT ETIENNE (FR)

(72) Inventors: Fabrizio ROMANO, BEYNOST (FR); Aurélien BERNARD, SAINT ETIENNE (FR)

(21) Appl. No.: 18/559,169

(22) PCT Filed: May 6, 2022
(86) PCT No.: PCT/EP2022/062236

§ 371 Date: Nov. 6, 2023

(30) Foreign Application Priority Data
May 6, 2021 (FR) FR2104803

(51) Int. Cl. A61F 9/009; A61B 90/00

(52) U.S. Cl. CPC A61F 9/009; A61B 2090/036; A61B 2218/007

(57) ABSTRACT

A coupling interface between a laser source and a tissue to be treated, remarkable in that the coupling interface comprises:
a ring (3) including:
   a double-walled partition (31),
   a window (33) transparent to a laser beam generated by the laser source, said window being sealingly mounted on the distal end of the ring so as to close the distal end of the ring,
a gel block (4) intended to be positioned in a working housing of the ring.

### TECHNICAL FIELD

The present invention relates to the technical field of surgical operations performed by laser, and more particularly to the field of ophthalmic surgery in particular for corneal or crystalline cutting applications.

The invention relates to an adaptation interface for coupling a laser source to human or animal tissue to be treated, such as a cornea or a crystalline lens.

By “laser source” is meant a light source capable of emitting a laser beam in the form of ultra-short pulses, whose duration is comprised between 1 femtosecond and 100 picoseconds, preferably comprised between 1 and 1000 femtoseconds, in particular in the order of one hundred femtoseconds.

### PRIOR ART

It is known from the state of the art to perform surgical operations of the eye by means of a laser source, such as corneal or crystalline cutting operations.

A laser source is an instrument capable of cutting the corneal tissue, for example, by focusing a laser beam in the stroma of the cornea, and by making a succession of small adjacent cavitation bubbles, which then form a cutting line.

More precisely, during the focusing of the laser beam in the cornea, a plasma is generated by non-linear ionization when the intensity of the laser source exceeds a threshold value, called optical breakdown threshold. A cavitation bubble is then formed, causing a very localized disruption of the surrounding tissues. Thus, the volume actually ablated by the laser source is very small compared to the disrupted area.

In order to allow the coupling of the laser source to a patient's eye, it is known to use an adaptation interface disposed between the eye of the patient and the laser source.

Such a coupling interface makes it possible to hold the eyeball in a stable and constant position, centered and at a known distance from the laser source, while avoiding any movement, throughout the duration of a treatment.

### 1. COUPLING INTERFACE KNOWN IN THE PRIOR ART

### 1.1. First Example of an Existing Coupling Interface

### 1.1.1. General Description

FIG. 1 illustrates a first example of a coupling interface of the prior art. The coupling interface comprises a ring including a truncated cone 1 of axis A-A′. The truncated cone 1 is open at its both ends. The large base 11 of the truncated cone 1 is intended to receive the end of the laser source. The small base of the truncated cone 1 is intended to come into contact with the eye 2. The coupling interface also comprises:
an annular gas flow groove 12 extending on the periphery of the small base, and
a tubular access member 122 extending radially outwardly and allowing the connection of the annular groove 12 to an outer aspiration device (not represented) via a connection nozzle 123.

The annular groove 12 has a U-shaped section defined by two circular rims 124, 125 intended to be pressed against the patient's eye 2. The aspiration device is known per se to those skilled in the art and allows creating a vacuum in the annular groove 12 once the circular rims 124, 125 are applied to the eye 2. The generation of a vacuum in the annular groove 12 allows securing the coupling interface on the eye 2 by suction effect throughout the entire duration of the treatment.

### 1.1.2. Principle of Operation of the First Example of an Existing Coupling Interface

The principle of operation of such a coupling interface is as follows.

At first, the practitioner places the ring of the coupling interface on the eye 2.

Once the ring is correctly positioned and centered, the aspiration device is activated to generate a vacuum in the space defined between the annular groove 12 and the eye 2 in order to secure the coupling interface on the eye 2 by suction.

When the ring is secured to the eye 2, the cone 1 is filled with a liquid having a refractive index close to that of the cornea until immersion of the cornea. The liquid thus immersing the cornea, has the advantage of simplifying the trajectory of the laser beams through the cornea by optically erasing the corneal diopter.

The laser source is then placed above the coupling interface, the distal portion of the laser source being inserted and locked on the coupling interface, so that the eye 2 and the optical axis of the laser are aligned and securely held throughout the duration of the treatment.

### 1.1.3. Disadvantages of the First Example of an Existing Coupling Interface

Such a coupling interface has many disadvantages.

### 1.1.3.1. Pressure Exerted on the Interface to Ensure the Sealing

The efficiency of the suction of the coupling interface on the eye 2 depends on the quality of the sealing between the circular rims 124, 125 and the eye 2.

A perfect contact must therefore be obtained over the entire circumference of the line of contact of each circular rim 124, 125.

This is not always simple, the patients' eyes being all different, the surface of the eyeball does not necessarily follow the slope between each circular rim 124, 125.

In order to overcome this inhomogeneity between the eyes of the patients, it is therefore necessary to strongly press the ring of the coupling interface to obtain a good sealing between the groove 12 and the eye 2, which is detrimental to the patient's comfort and safety. Indeed, the force exerted on the ring of the coupling interface to obtain a satisfactory sealing tends to enter the circular rims 124, 125 in the conjunctiva.

The force to be applied for achieving the sealing is sometimes so significant that the discomfort of the patient can tend to be painful. Moreover, this pressure, even though it is of short duration, immediately causes the increase of the intraocular pressure of the patient which, in some patients at risk (high myopia, retinal detachment history or glaucoma), may result in serious side effects.

### 1.1.3.2. Level of the Vacuum

The level of the vacuum must be significant in order to hold the coupling interface in position.

Indeed, in order to resist the ocular micro-movements and the tendency to un-suction, the aspiration force must be significant.

However, it is known that the aspiration force F is a function of the Vacuum P and the Surface S on which this vacuum is applied, namely:

$$F = P \times S .$$

In traditional coupling interfaces, the surface on which the vacuum is applied is small (i.e. limited to the surface of the eye between the two circular rims), it is necessary to apply a vacuum of significant value to obtain an aspiration force F allowing to resist the ocular micro-movements and the tendency to un-suction.

The importance of the applied vacuum sometimes leads to the appearance of conjunctival hemorrhage (effusion of red blood cells linked to the aspiration under the conjunctiva). Although superficial ecchymoses are generally very limited, and have no effect on vision, they are always a source of concern for patients and are quite unattractive, and it is preferable to limit their appearance.

### 1.1.3.3. Cost

Some traditional coupling interfaces work with specific surgical equipment. These had to integrate a suction system with pump, nozzles and vacuum sensor. Each system being specific, it is necessary to buy all these accessories. The present invention avoids to make this unnecessary expense. Indeed, this patient interface works with accessories already present in the operating room and in particular the phacoemulsification apparatus. The latter is equipped with an aspiration pump with vacuum sensor and an infusion line, and already has adequate nozzles. Our experimental tests demonstrate that the new coupling interface, object of this invention, works perfectly with a phacoemulsification apparatus, which generates a substantial saving for the customer.

### 1.2. Second Example of an Existing Coupling Interface

In order to overcome the aforementioned drawbacks, the document WO2018154038 proposes a coupling interface comprising:
a ring including a side wall, a proximal end for contacting the tissue to be treated, and a distal end for receiving the end of the laser source,
a window transparent to a laser beam generated by the laser source, said window being sealingly mounted on the ring so as to close the distal end of the ring,
an aspiration through channel extending between the inner and outer faces of the side wall, the aspiration channel being intended to be connected to an aspiration module for allowing the generation of a vacuum in an interior space defined between the window, the side wall, and the upper part of the eyeball,
an irrigation through channel extending between the inner and outer faces of the side wall, the irrigation channel being intended to be connected to an irrigation module for allowing the infusion of liquid into the interior space.

By generating a vacuum throughout the interior space of the coupling interface, the surface area over which the vacuum acts is greatly increased compared to the coupling interface shown in FIG. 1 (in which the vacuum is applied only at a circular rim). This reduces the level of vacuum applied to the eye, which contributes to a reduction in side effects due to too high vacuum. The coupling procedure becomes more pleasant for the patient.

Although this interface has many advantages, the patient's eye can move by one or several tens of microns. However, when performing certain cuts, it is preferable for the cornea to remain completely immobilised.

The document DE 10 2005 040338 describes an adapter for mechanically coupling a laser treatment device to a patient's eye. The adapter according to DE 10 2005 040338 comprises: a glass lens body having a lower face adapted to the curvature of the human cornea, and a holder surrounding the lens body.

The document WO 2019/104256 describes a patient interface for an adjustable intraocular lens irradiation system.

An object of the present invention is to provide a new coupling interface with the same advantages as the coupling interface described in WO2018154038, and allowing the patient's eye to be completely immobilised (eye movement of less than ten microns).

### DISCLOSURE OF THE INVENTION

For this purpose, the invention proposes a coupling interface between a laser source and a tissue to be treated, remarkable in that the coupling interface comprises:
a ring having a proximal end for contacting the tissue to be treated and an opposite distal end, the ring including:
   a double-walled partition comprising:
      an inner (cylindrical) side wall having an upper edge and a lower edge and
      an outer (cylindrical) side wall having an upper edge and a lower edge, the lower edge of the inner side wall being closer to the distal end than the lower edge of the outer side wall, and the upper edges of the inner and outer side walls being closer to the distal end than the lower edges of the inner and outer side walls,
   a perforated disc extending between the lower edges of the inner and outer side walls, the disc including through lumens,
   a window transparent to a laser beam generated by the laser source, said window being sealingly mounted to the upper edges of the inner and outer side walls so as to close the distal end of the ring, the window defining:
      an aspiration chamber peripheral to the perforated disc, the inner side wall and the outer side wall, and
      a working housing centered with the inner side wall, and
   an aspiration channel extending between the inside and outside faces of the outer side wall, so as to open into the aspiration chamber (between the inner and outer walls), the aspiration channel being intended to be connected to an aspiration module for allowing the generation of a vacuum in an interior space defined between the aspiration chamber,
a gel block intended to be positioned in the working housing, said gel block having:
   a side flank with a shape complementary to the shape of the inner side wall, the side flank being intended to come into contact with the inside face of the inner side wall,
   a substantially planar circular upper base, the upper base being intended to come into contact with the window,
   a circular and concave lower base, the lower base being intended to come into contact with the tissue to be treated.

Advantageously, the gel block is removably mounted in the working housing. Moreover, the gel block is capable of sliding along the inner side wall of the double partition. Finally, the gel block is deformable.

The coupling interface described above differs from existing interfaces, in particular in that it comprises a ring including a double-walled partition, a window mounted on the upper edges of the double-walled partition, a working housing delimited by the window and the inner side wall of the double-walled partition, and a gel block removably mounted in the working housing.

The combination of these features makes it possible to have a coupling interface that conforms to the shape of the eye of the patient, unlike for example the adapter according to DE 10 200 5 040 338 (which proposes the use of a glass lens body) and in which it is the eye of the patient that is deformed (via the application of a large bearing force) to conform to the shape of the lower face of the glass lens body.

The use of a gel block allows the risk of displacement of the patient's eye during the surgical procedure to be reduced. Indeed, the coefficient of friction of the contact surface between the eye and the gel block is greater than the coefficient of friction of the contact surface between the eye and the liquid with a refractive index close to that of the cornea, as proposed in document WO2018154038. The use of a gel block also allows the risks of vertical displacements of the cornea to be reduced (hydrogel limits vertical movements better than fluids). This is very important for non-transfixing corneal incisions (such as arcuate incisions for the correction of astigmatism).

However, the present invention is not limited to replacing a coupling liquid with a gel block. Indeed, in order to benefit from the advantages associated with the device described in WO2018154038, the inventors have had to rethink the entire structure of the coupling interface according to WO2018154038.

In particular, the presence of a double-walled partition allows the gel block to be prevented from being aspirated through the aspiration channel. It also ensures that the gel block is centred when the patient interface is fitted.

In addition, the combination of the double-walled partition and the gel block makes it possible to reduce discomfort for the patient compared with the solutions of the prior art as illustrated in FIG. 1. In fact, in the interface illustrated in FIG. 1, two circular rims 124, 125 (i.e. an inner rim 124 and an outer rim 125) are intended to come into contact with the patient's eye, which requires the practitioner to press hard on the ring to ensure perfect contact between the patient's eye and each of these circular rims 124, 125. In the context of the present invention, the gel block replaces the inner rim. Thus, the double-walled partition has a single rim intended to come into contact with the patient's eye and:
a perfect contact between the gel block and the patient's eye is achieved thanks to the deformability of the gel block, and
a perfect contact between the single rim of the double-walled partition and the patient's eye does not require the user to apply a significant bearing force (it is easier to obtain perfect contact between the eye and a single circular rim—principle of the ring on the sphere where 360° contact is always obtained—rather than between the eye and two concentric rims as proposed with the prior art patient interface illustrated in FIG. 1).

More specifically, to set up the patient interface illustrated in FIG. 1, it is the patient's eye that is deformed (via the application of a significant bearing force) to conform to the shape of the patient interface: on the contrary with the solution of the present invention, it is the patient interface that conforms to the shape of the patient's eye thanks to the use of:
a double-walled partition with a single rim of contact with the patient's eye, and the use of
a deformable mobile gel block.

Such a coupling interface allows rapid and painless suction while guaranteeing the immobility of the patient's eye. This coupling interface is compatible with the use of an aspiration module, which can be integrated into an apparatus commonly found in the ophthalmic operating room: the phacoemulsifier or any other aspiration module with adequate performance equipped with the necessary sensors. Of course, the present invention is also compatible with the use of a non-integrated aspiration module.

Several models may be provided for the gel block, the concave circular lower base of each gel block model having a radius of curvature different from the radii of curvature of the concave circular lower bases of the other gel block models.

Indeed:
the deformability of the gel block being limited (elastic modulus between 0.1 and 1 MPa), and
the size of an eye being variable from one patient to another,
   it may be advantageous to have several models of gel blocks to allow optimum adaptation of the gel block to all eye sizes, without having to constrain the patient's eye.

For this reason, the inventors have developed three gel block models:
a first model of gel block with a circular concave lower base having a radius of curvature of 7.1 millimetres,
a second model of gel block with a circular concave lower base having a radius of curvature of 7.6 millimetres,
a third model of gel block with a circular concave lower base having a radius of curvature of 8.1 millimetres.

Of course, the reader will appreciate that more (or less) than three gel block models can be provided. In all cases, the choice of the most suitable gel block model for a given patient can be made by the practitioner on the basis of standard pre-operative eye measurements.

Preferred but non-limiting aspects of the coupling interface are as follows:
the gel block may be made of a material having an elastic modulus of between 0.1 and 1 MPa;
the height of the gel block may be substantially equal to the height of the inner side wall of the double-walled partition;
the double-walled partition and the gel block may be cylindrical, the diameter of the gel block being substantially equal to the diameter of the inner side wall of the double-walled partition, for example between 11 and 14 millimetres;
the lower base of the gel block may be concave, the distance between the upper and lower bases:
   ranging between 1 millimetre and 2.5 millimetres at the centre of the lower base,
   being substantially equal to 4.5 and 6 millimetres at the periphery of the lower base:
the lower base of the gel block may be concave, the radius of curvature of the lower base being between 6.5 and 8.5 millimetres;
the height of the outer side wall may be greater than the height of the inner side wall so that the lower edge of the inner side wall is closer to the window than the lower edge of the outer side wall, the perforated disc connecting the lower edges of the inner and outer side walls having a substantially frustoconical shape so that the axes of the lumens are oriented towards a longitudinal axis of the ring;
the surface area of the perforated disc covered by the through lumens may be greater than the surface area of the perforated disc not covered by the through lumens;
the ring may further comprise a frustoconical flange open at its largest diameter rim, said flange being connected to the upper edge of the outer side wall by its smallest diameter rim;
the ring may further comprise an annular brim integral with the largest diameter rim, said brim extending radially outwards and including a flexible rubber layer on its face opposite the flange;
the double-walled partition, the flange and the annular brim may be in one-piece.

The invention also relates to a method for installing a coupling interface between a laser source and a tissue to be treated, the coupling interface comprising
a ring including:
   a double-walled partition having inner and outer side walls,
   a perforated disc extending between lower edges of the inner and outer side walls,
   a window transparent to a laser beam mounted to the upper edges of the inner and outer side walls, the inner and outer side walls, the perforated disc, and the window defining an aspiration chamber of the ring, and the window and the inner side wall also defining a working housing of the ring,
a gel block including a side flank and upper and lower bases,
   the method comprising the following steps:
inserting the gel block into the working housing,
positioning the ring containing the gel block above the tissue to be treated (centering),
contacting:
   the lower edge of the outer side wall with the tissue to be treated at a region peripheral to said tissue to be treated, and
   the lower base of the gel block with the tissue to be treated at a central region of said tissue to be treated,
activating an aspiration device to create a vacuum in the aspiration chamber, between the inner side walls of the ring and the side flank of the gel block,
deactivating the aspiration device as soon as the desired vacuum threshold is reached,
holding the constant vacuum throughout the entire duration of the procedure.

Once the procedure is completed, the coupling interface is separated from the tissue to be treated by restoring the atmospheric pressure in the aspiration chamber.

### SHORT DESCRIPTION OF THE DRAWINGS

Other characteristics and advantages of the invention will clearly emerge from the description which is carried out hereinafter, for illustrative and non-limiting purposes, with reference to the appended figures, in which:

FIG. 1 is a schematic representation of a coupling interface of the prior art:

FIGS. 2 and 3 are schematic representations of a coupling interface according to the invention,

FIG. 4 illustrates a method for installing the coupling interface according to the invention.

### DETAILED DISCLOSURE OF THE INVENTION

The coupling interface according to the invention will now be described with reference to the figures. In these different figures, the equivalent elements are designated by the same reference numeral.

The coupling interface is intended to be disposed between a laser source and a target to be treated 2. The target 2 is for example a human or animal tissue to be treated such as an eyeball and more specifically a cornea or a crystalline lens.

In the remainder of the description, the invention will be described, by way of example, for the treatment of a cornea of a human or animal eye. Nevertheless, it is obvious to those skilled in the art that the coupling interface according to the invention can be used in other applications.

### 1. General Description of the Coupling Interface

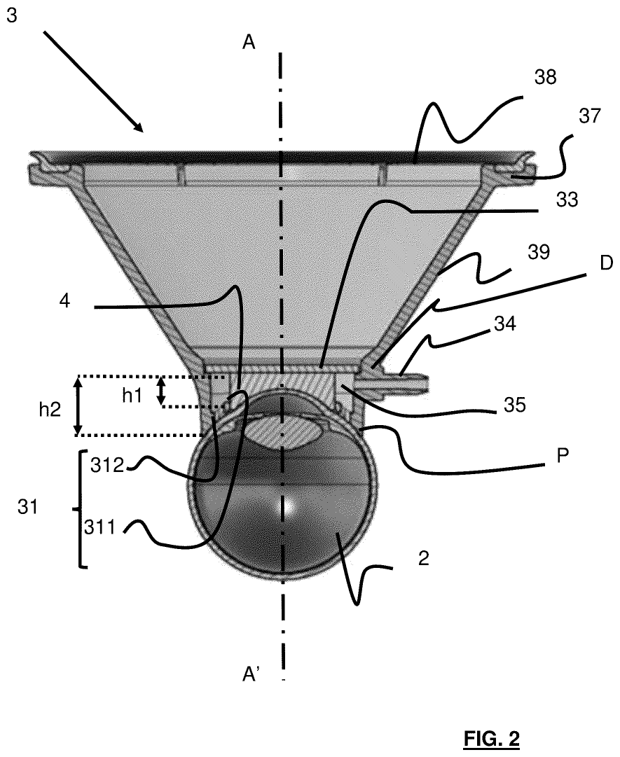

With reference to FIG. 2, the coupling interface comprises:
a ring 3 extending along a longitudinal axis A-A′ and having proximal P and distal D ends, and
a gel block 4 having a generally cylindrical shape.

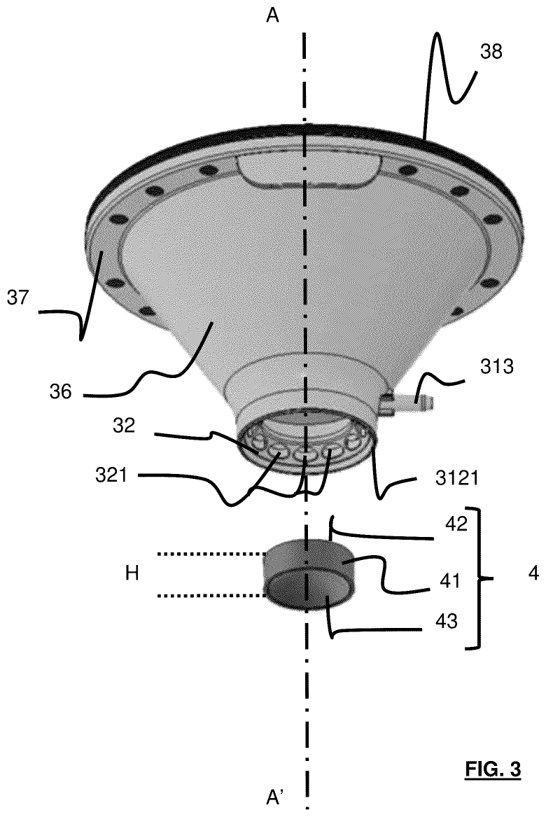

The ring 3 comprises: a double-walled partition 31 having inner and outer walls 311, 312, a perforated disc 32 (FIG. 3) at one of the ends of the partition 31, a window 33 transparent to the laser radiation at the other end of the partition 31, and an aspiration channel 34 opening between the walls 311, 312 of the partition 31. The double-wall partition 31 is arranged such that only the lower edge 3121 of the outer wall 312 protrudes outwards of the ring 3. This lower edge 3121 forms the proximal end P of the ring 3. More specifically, the lower edge 3121 of the outer side wall 312 constitutes the single ridge of the ring 3 intended to come into contact with the eye 2 of the patient at the periphery of the globe.

The window 33 is sealingly mounted on the distal end D of the ring 3 so as to close the distal end D. The ring 3 is therefore open only at its proximal end P intended to come into contact with the eye 2 of the patient.

The partition 31, the disc 32 and the window 33 delimit two spaces inside the ring:
a circular aspiration chamber 35 in which vacuum can be created by connecting an aspiration unit to the aspiration channel 34, and
a working housing 36 adapted to receive the gel block 4, and through which the laser beams emitted by a laser source are applied to the tissue 2 to be treated.

To fix the coupling interface on the patient's eye 2, the gel block 4 is mounted in the working housing 36, the ring 3 and the gel block 4 are positioned on the eye 2 of the patient, and vacuum is made in the aspiration chamber 35.

Prior to application of the vacuum, the eye 2 of the patient is in contact with the patient interface:
at the gel block 4, and
at the lower edge 3121 of the outer side wall 312 (which constitutes the single ridge in contact with the eye 2 of the patient).

Thus and unlike the existing coupling interfaces, the attachment of the coupling interface on the eye 2 by the generation of a vacuum, does not require a perfect bearing between two circular rims defining an annular groove.

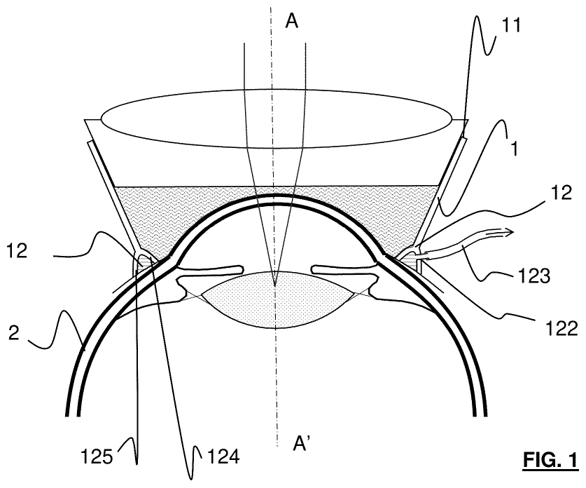

Due to this new design (i.e. single circular rim 3121 and presence of a deformable gel block 4), the generation of the vacuum is simplified and more pleasant for the patient since it is the coupling interface that conforms to the configuration of the eye (via the deformation of the gel block) and not the eye that conforms to the configuration of the patient interface (as is the case with the device illustrated in FIG. 1).

The coupling interface according to the invention will now be described according to the invention.

### 2. DETAILED DESCRIPTION OF THE COUPLING INTERFACE

### 2.1. Ring

With reference to FIGS. 2 and 3, the ring 3 comprising the double-walled partition 31, the perforated disc 32, the transparent window 33 and the aspiration channel 34 have been illustrated.

### 2.1.1. Double-Walled Partition

The double-walled partition 31 has a generally cylindrical shape, and is open at both ends.

More specifically, the double-walled partition 31 is composed of an inner side wall 311 and an outer side wall 312. Each side wall 311, 312 includes respective upper and lower edges. As previously described, the lower edge 3121 of the outer wall 312 is intended to come into contact with the eye 2 of the patient. Therefore, the lower edge 3121 of the outer side wall 312 may advantageously be flared to best conform to the shape of an eye 2. The flared lower edge 3121 of the outer wall 312 may (for example) have a generally frustoconical shape curved outwards capable of being applied to the outer surface of the eye 2 in a non-traumatic manner. With reference to FIG. 2, this flared lower edge 3121 has a concave profile of a radius of curvature substantially equal to the radius of curvature of the eye 2 to allow a tangential bearing.

As illustrated in FIG. 3, the lower edge of the inner side wall 311 is recessed inwards of the ring 3. In particular, the lower edge 3121 of the outer side wall 312 protrudes outwards, while the lower edge of the inner side wall 311 is offset along the longitudinal axis A-A′ inwards of the ring 3.

In particular, the height h1 of the inner side wall 311 is less than the height h2 of the outer side wall 312, so that the lower edge of the inner side wall 311 is closer to the window 33 than the lower edge 3121 of the outer side wall 312.

Thus, the coupling interface includes a single circular ridge intended to come into contact with the eye 2 of the patient. The fact that the coupling interface comprises a single circular ridge of contact (rather than two circular rims as proposed in the traditional coupling interfaces as illustrated in FIG. 1) makes it easy to achieve a perfect contact over the entire circumference of the contact line between the eye 2 and the ring 3.

### 2.1.2. Perforated Disc

The perforated disc 32 enables the inner and outer side walls 311, 312 of the double-wall partition 31 to be connected. It comprises through lumens 321 or recesses intended to cooperate with the sclera of the eye 2 of the patient when the coupling interface is positioned on the eye 2 of the patient and that a vacuum is generated in the aspiration chamber 35, so that the conjunctival membrane is slightly aspired into these recesses, thus forming an integral assembly reducing the risk of un-suction.

In the embodiment illustrated in FIG. 3, the perforated disc 32 has a substantially frustoconical shape such that the axes of the lumens of the disc 32 are oriented inwards of the ring 3. Of course, the perforated disc 32 may have other shapes. For example, the perforated disc 32 may have the form of a truncated torus. In this case, the radius of curvature of the perforated disc 32 is selected from a range between 5 and 10 millimeters, in order to optimize the conformation of the disc 32 to the eye 2 of the patient.

The number and the shape of the through lumens 321 may vary depending on the intended application. Preferably, the surface area of the disc 32 covered by the lumens 321 is greater than the surface area of the disc 32 not covered by the lumens 321. This makes it possible to maximize the surface of the eye on which the aspiration force is applied, and therefore to minimize the vacuum level (i.e. the intensity of the vacuum) necessary to ensure that the coupling interface is held in position on the eye 2 of the patient.

### 2.1.3. Window

With reference to FIG. 2, the window 33 transparent to the beam generated by the laser source is sealingly mounted to the upper edges of the inner and outer side walls 311, 312 of the double-walled partition 31.

The window 33 has the shape of a disk. Of course, the window 33 may have other shapes (square, rectangular, elliptical shapes) depending on the intended application.

The window 33 may be designed in different materials such as glass or plastic (polycarbonate, poly(methyl methacrylate), etc.).

In the embodiment illustrated in FIGS. 2 and 3, the window 33 and the double-walled partition 31 are made in two separate parts. Nevertheless, in some embodiments, the window 33 and the double-walled partition 31 can be made in one part (monoblock). This allows limiting the risks of leakage at the junction between the double-walled partition 31 and the window 33.

When the window 33 and the double-walled partition 31 are two separate parts of the ring 3, the window 33 can be attached to the double-walled partition 31 by gluing, welding or any other technique allowing sealingly attaching the window 33 to the double-walled partition 31.

The window 33 may be anti-reflective or have any other type of optical treatment in order to improve the transmission of the laser beam according to its wavelength.

### 2.1.4. Aspiration Channel

The ring 3 also comprises a tubular through channel 34 formed in the outer side wall 312 of the double-walled partition 31. This channel, referred to as the “aspiration channel”, opens between the inner and outer walls 311, 312 of the double-walled partition 31, and extends radially outwards perpendicularly to the axis A-A′.

The aspiration channel 34 allows the connection of a remote device to the coupling interface via a nozzle. In particular, the aspiration channel 34 allows the coupling interface to be connected to an aspiration device for generating a vacuum in the aspiration chamber 35 defined between the window 33, the inner and outer side walls 311, 312, and the perforated disc 32.

The presence of a single channel 34 limits the footprint of the coupling interface and reduces the number of nozzles and connectors.

### 2.1.5. Other Optional Elements of the Ring

In addition to the elements described above, the ring 3 may also comprise a frustoconical flange 39 connected to the upper edge of the outer side wall 312 by its smallest diameter rim.

Such a flange 39 is open at its largest diameter rim, and is integral with an annular brim 37 extending radially outwards of the flange 39.

Advantageously, this annular brim 37 comprises a layer of flexible rubber 38 on its face opposite the flange 39. This allows the laser source to be easily fixed to the coupling interface by suction. Indeed, even if several solutions (mechanical, magnetic, etc.) can be envisaged in order to secure the laser source to the coupling interface, it is preferable to use means of securing by vacuum in order to limit the risks of “jerking” during this securing process.

### 2.2. Gel Block

The coupling interface also comprises a gel block 4 intended to cooperate with the ring 3.

With reference to FIG. 3, the gel block 4 has a generally cylindrical shape. It comprises:
a side flank 41 having a shape complementary to the shape of the inner side wall,
a substantially planar circular upper base 42,
a concave circular lower base 43.

The gel block 4 is intended to be positioned in the working housing 36 of the ring 3. More specifically, the side flank 41 of the gel block 4 is intended to come into contact with the inside face of the inner side wall 311, and the upper base 42 is intended to come into contact with the window 33, while the lower base 43 is intended to come into contact with the eye 2 of the patient.

This is why the lower base 43 has a concavity defined by a radius of curvature chosen in a range of between 5 and 10 millimetres, preferably between 6.5 and 8.5 millimetres. This optimizes the conformation of the lower base to the eye 2 of the patient.

Advantageously, the gel block 4 may be a hydrogel or any other biocompatible and sterilizable transparent flexible material, with optical properties compatible with the propagation of a femtosecond laser. For example, the hydrogel may comprise a mixture of fluorosilicone and hydrophilic monomers. Various hydrogel variants may be used, such as hydrogels having a water content greater than 70%, or between 50% and 70%, or between 30 and 50%. This makes it possible to have gel blocks having different lubricating and optical properties, as well as different compressibility values.

In all cases, the dimensions and the shape of the gel block 4 are determined so that the gel block 4 closely cooperates with the working housing 36.

In particular, the height H of the gel block 4 is preferably substantially equal to or slightly greater than the height h1 of the inner side wall 311. This makes it possible to limit the risks of movement of the gel block towards the aspiration chamber 35 during the generation of the vacuum, the gel block being compressed between the tissue and the window during the generation of the vacuum. Thus, the risks of decentering of the gel block are reduced. This also avoids the presence of bubbles:
between the gel block 4 and the window 33 and/or
between the gel block 4 and the eye 2 of the patient, once the coupling interface is pressed against on the eye 2 to be treated.

Furthermore, the diameter of the gel block 4 is preferably substantially equal to or slightly smaller than the diameter of the inner side wall 311 (between 11 and 14 millimeters). This makes it possible to facilitate the insertion of the gel block into the working housing by ensuring its mobility in translation along the axis A-A′.

Finally, in order for the gel block 4 to hold the eye 2 of the patient in position, the distance between the upper and lower bases of the gel block 4 can:
range between 1 millimeter and 2.5 millimeters at the center of the circular lower base,
substantially equal to 4.5 and 6 millimeters at the periphery of the lower base.

### 3. PRINCIPLE OF OPERATION

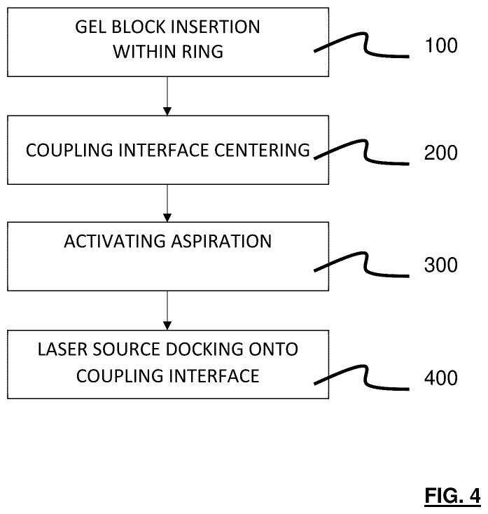

The principle of operation of the coupling interface according to the invention will now be described in more detail with reference to the coupling interface represented in FIGS. 2 and 3, and to the method illustrated in FIG. 4.

It is assumed that the aspiration channel 34 has been previously connected to the aspiration device (not represented) via a nozzle.

In a first step 100, the practitioner inserts (under sterile conditions) the gel block 4 into the working housing 36 and pushes it at the bottom of the housing until the distal planar portion of the gel block adheres to the window 33.

In a second step 200, the practitioner positions the ring 3 and the gel block 4 above the eye 2, so that the center of the eye and the assembly composed of the ring and the gel block 4 are aligned. When the coupling interface (ring+gel block) is centered on the eye of the patient, the practitioner contacts the lower edge 3121 of the outer side wall 312 with the periphery of the eye 2 of the patient and more precisely on a surface area of the globe covered by the conjunctival membrane.

When the coupling interface is properly centered on the eye 2 of the patient, the practitioner activates the aspiration device (step 300). The activation of the aspiration device allows creating a vacuum in the aspiration chamber 35 which applies the suction. Due to the generation of the vacuum, the conjunctival membrane covering the sclera of the eye 2 is aspired at the through lumens 321 of the perforated disc 32. This limits relative movements of the eye 2 relative to the coupling interface. The orientation of the through-lumens 321 (the axes of symmetry of which are oriented towards the axis A-A′) allows the application of a tangential force to the eye 2 of the patient tending to immobilize the latter.

The eye 2 presses against the lower edge 3121 of the outer side wall 312 on the one hand, and against the gel block 4 on the other hand. Due to its malleability, the gel block perfectly conforms to the shape of the eye 2 of the patient.

Once the coupling interface is sucked onto the eye 2, the practitioner controls docking of the laser source on the coupling interface, while maintaining the aspiration device activated (step 400). The fixation of the laser source (which may for example be mounted on an articulated arm as described in document WO 2019/145487) at the coupling interface may advantageously be made by suction to limit any unexpected movement of the coupling interface.

### 4. CONCLUSIONS

The surgical procedures performed in ophthalmology and using a laser source (in particular femtosecond laser) typically use a system for holding the eyeball, which must be active throughout the entire duration of exposure of the patient to the laser beam.

Indeed, the risk would be that, in case of unexpected and uncontrolled movement of the eyeball, the beam reaches areas not supposed to be affected and generates more or less serious lesions of the intraocular structures.

The above-described coupling interface allows optimal holding of the eyeball, and provides the practitioner with accurate knowledge of the position in the space of the eyeball to accurately direct the laser beam to its target.

The reader will understand that many modifications can be made to the invention described above without physically departing from the new teachings and advantages described herein.

1. A coupling interface between a laser source and a tissue to be treated, wherein said coupling interface comprises:
a ring which has a proximal end configured to contact the tissue to be treated and an opposite distal end and which includes:
a double-walled partition which has:
an inner side wall which includes an upper edge and a lower edge, and
an outer side wall which includes an upper edge and a lower edge,
wherein the lower edge of the inner side wall is closer to the distal end than the lower edge of the outer side wall, and wherein the upper edges of the inner and outer side walls is closer to the distal end than the lower edges of the inner and outer side walls,
a perforated disc which extends between the lower edges of the inner and outer side walls, wherein the perforated disc includes through lumens,
a window sealingly mounted to the upper edges of the inner and outer side walls so as to close the distal end of the ring, wherein said window is transparent to a laser beam generated by the laser source and defines:
an aspiration chamber peripheral to the perforated disc, the inner side wall and the outer side wall, and
a working housing disposed centrally with the inner side wall, and
an aspiration channel extending between the inside and outside faces of the outer side wall, so as to open into the aspiration chamber, wherein the aspiration channel is connected to an aspiration module for allowing the generation of a vacuum in an interior space defined by the aspiration chamber,
a gel block removably mounted in the working housing, wherein said gel block is able to slide along the inner side wall of the double partition and has:
a side flank with a shape complementary to the shape of the inner side wall, wherein the side flank is in contact with the inside face of the inner side wall,
a substantially planar circular upper base, the upper base is in contact with the window,
a lower base, wherein the lower base is in-contact with the tissue to be treated.

2. The coupling interface according to claim 1, wherein the height of the gel block is substantially equal to the height of the inner side wall of the double-walled partition.

3. The coupling interface according to claim 1, wherein the double-walled partition and the gel block are cylindrical, and wherein the diameter of the gel block is substantially equal to the diameter of the inner side wall of the double-walled partition.

4. The coupling interface according to claim 1, wherein the lower base of the gel block is concave, and wherein the distance between the upper base (42) and the lower base (43):
ranges between 1 millimetre and 2.5 millimetres at the centre of the lower base,
is substantially equal to 4.5 and 6 millimetres at the periphery of the lower base.

5. The coupling interface according to claim 1, wherein the lower base of the gel block is concave, and wherein the radius of curvature of the lower base ranges between 6.5 and 8.5 millimetres.

6. The coupling interface according to claim 1, wherein the height of the outer side wall is greater than the height of the inner side wall so that the lower edge of the inner side wall is closer to the window than the lower edge of the outer side wall, and wherein the perforated disc connecting the lower edges of the inner and outer side walls (311, 312) have a substantially frustoconical shape so that the axes of the lumens are oriented towards a longitudinal axis of the ring.

7. The coupling interface according to claim 1, wherein the surface area of the perforated disc covered by the through lumens is greater than the surface area of the perforated disc not covered by the through lumens.

8. The coupling interface according to claim 1, wherein the ring further comprises a frustoconical flange open at its largest diameter rim, and wherein said flange is connected to the upper edge of the outer side wall by its smallest diameter rim.

9. The coupling interface according to claim 8, wherein the ring further comprises an annular brim integral with the largest diameter rim, and wherein said brim extends radially outwards and includes a flexible rubber layer on its face opposite the flange.

10. The coupling interface according to claim 9, wherein the double-walled partition, the flange and the annular brim are in one piece.
